(19) United States
(12) Patent Application Publication
(10) Pub. No.: US 2020/0163580 A1
(43) Pub. Date: May 28, 2020

(54) ANKLE COIL

(71) Applicant: Siemens Healthcare GmbH, Erlangen (DE)

(72) Inventor: Wei Ge Bao, Shenzhen (CN)

(73) Assignee: Siemens Healthcare GmbH, Erlangen (DE)

(21) Appl. No.: 16/689,408

(22) Filed: Nov. 20, 2019

(30) Foreign Application Priority Data
Nov. 23, 2018 (CN) 201811406791.2

(51) Int. Cl. A61B 5/055; A61B 5/00; G01R 33/34

(52) U.S. Cl. CPC A61B 5/0555; G01R 33/34084; A61B 5/4595

(57) ABSTRACT

An ankle coil is disclosed that includes a first coil portion and a second coil portion. The first coil portion has a first channel that is hollow and extends in a first extension direction. The second coil portion has a second channel that is hollow and extends in a second extension direction intersecting with the first extension direction. The second coil portion is rotatably arranged on the first coil portion, with an axis of rotation perpendicular to both the first extension direction and the second extension direction, and notches are provided at a joint between the first coil portion and the second coil portion. The ankle coil provided in the present disclosure in use allows an ankle to be placed in a naturally relaxed state, and can be snugly fitted with the foot, so that the comfort of a user is improved while ensuring the imaging quality.

### CROSS-REFERENCE TO RELATED APPLICATIONS

The present application claims the benefit of the filing date of China patent application no. 201811406791.2, filed on Nov. 23, 2018, the contents of which are incorporated herein by reference in their entirety.

### TECHNICAL FIELD

The present disclosure relates to an ankle coil, and in particular to an ankle coil which is angle adjustable to accommodate natural plantar flexion of an ankle.

### BACKGROUND

Currently, flexible coils or rigid ankle coils are commonly used in clinical magnetic resonance scanning of ankle joints. The flexible coils require, when in use, an additional device to fix a foot in order to reduce imaging artifacts caused by involuntary movement, which leads to a complicated operation. The rigid ankle coils are complex in structure and wiring, and in order to obtain a better image quality during use, a foot of a user needs to be attached to the coil as closely as possible, which requires the user to keep the sole of the foot perpendicular to the shank. This tightens the foot of the user and results in discomfort, as the user cannot hold this position on his own for a long time. As a result, the comfort of the user is compromised, and movement artifacts are produced.

### SUMMARY

An objective of the present disclosure is to provide an ankle coil, which in use allows an ankle to be placed in a naturally-relaxed state, and can be snugly fitted with the foot so that the comfort of a user is improved while ensuring the imaging quality.

The present disclosure provides an ankle coil comprising a first coil portion and a second coil portion. The first coil portion has a first channel that is hollow and extends in a first extension direction. The second coil portion has a second channel that is hollow and extends in a second extension direction intersecting with the first extension direction. The second coil portion is rotatably arranged on the first coil portion, with an axis of rotation perpendicular to both the first extension direction and the second extension direction, and notches are provided at a joint between the first coil portion and the second coil portion.

The ankle coil provided in the present disclosure comprises the first coil portion and the second coil portion that are formed separately, one being used to accommodate a heel and a shank, and the other being used to accommodate a foot. The first coil portion is connected to the second coil portion in a rotational manner, thereby being able to accommodate plantar flexion angles of feet of different sizes, and also being able to be snugly fitted with the foot, so that the comfort of the user is improved while preserving the imaging quality. In addition, the first coil portion in the present disclosure can integrally surround the heel and the shank, which, compared with a case in which a bottom for supporting the shank and other coil portions should be arranged separately in an existing ankle coil and be assembled when in use, enables the user to wear and remove the ankle coil more conveniently, and enables imaging coil units to be arranged on all sides of the first coil portion surrounding the shank.

The ankle coil provided in the present disclosure comprises a first rigid coil portion and a second rigid coil portion with channels formed respectively, which cooperate with a first flexible coil portion and a second flexible coil portion to form accommodation spaces, one being used to accommodate a heel and a shank, and the other being used to accommodate a foot. The first rigid coil portion is connected to the second rigid coil portion in a rotational manner, and thereby is able to accommodate plantar flexion angles of feet of different sizes, and also is able to be snugly fitted with the foot. The comfort of the user is improved while preserving the imaging quality, and the layout of internal coils is facilitated.

In another exemplary embodiment of the ankle coil, the first coil portion comprises a first rigid coil portion and a first flexible coil portion. The first rigid coil portion is formed with a first channel having an opening on the side oriented in a direction of rotation thereof towards the second coil portion. The first flexible coil portion is arranged on the first rigid coil portion and is able to cover the side of the first channel that is oriented in the direction of rotation. The second coil portion comprises a second rigid coil portion and a second flexible coil portion. The second rigid coil portion is rotatably arranged on the first rigid coil portion, and is formed with a second channel having an opening opposite to the first channel in the direction of rotation. The second flexible coil portion is arranged on the second rigid coil portion and is able to cover the side of the second channel that is oriented in the direction of rotation. This allows the ankle coil to be snugly fitted with the foot, thus achieving better imaging quality.

In yet another exemplary embodiment of the ankle coil, the first rigid coil portion, the first flexible coil portion, the second rigid coil portion, and the second flexible coil portion are each provided with coil units for magnetic resonance imaging.

In yet another exemplary embodiment of the ankle coil, the ankle coil further comprises a hinge member which comprises a first connector, a second connector, and a hinge shaft. The first connector is fixed to the first rigid coil portion. The second connector is fixed to the second rigid coil portion. One of the first connector and the second connector is fixed to the hinge shaft, the other is rotatably sheathed on the hinge shaft, and an axial direction of the hinge shaft coincides with the axis of rotation. Therefore, a rotational connection between the first rigid coil portion and the second rigid coil portion is achieved by means of a simple structure.

In yet another exemplary embodiment of the ankle coil, the ankle coil further comprises a positioning member, wherein the positioning member is arranged in one of the first rigid coil portion and the second rigid coil portion so as to be movable along a direction parallel to the axis direction of rotation, and bears against the other of the first rigid coil portion and the second rigid coil portion along the direction of movement of the positioning member in order to fix a position of the second rigid coil portion relative to the first rigid coil portion by means of a frictional force. Therefore, movement artifacts caused by coil shaking due to involuntary movement of a patient are avoided.

In yet another exemplary embodiment of the ankle coil, the second rigid coil portion has a limiting portion, and when the second rigid coil portion is rotated towards the first rigid coil portion in the direction of rotation such that the extension direction of the second channel is perpendicular to the extension direction of the first channel, the limiting portion bears against the first rigid coil portion to prevent further rotation of the second rigid coil portion. Therefore, a case in which an angle between the first rigid coil portion and the second rigid coil portion is too small to facilitate the user to put the ankle into the ankle coil is avoided.

In yet another exemplary embodiment of the ankle coil, the ankle coil further comprises an elastic member, one end of the elastic member applies a force to the first rigid coil portion, and the other end applies a force to the second rigid coil portion so as to continuously apply, to the second rigid coil portion, an elastic force (e.g. a biasing force) that rotates the second rigid coil portion towards the first rigid coil portion in the direction of rotation. Therefore, the second rigid coil portion can be automatically restored to a position at which the extension direction of the second channel is perpendicular to the extension direction of the first channel, so that the user can put the ankle into the ankle coil.

In yet another exemplary embodiment of the ankle coil, the ankle coil further comprises a damping member which is arranged between the first rigid coil portion and the second rigid coil portion to increase resistance against rotation of the second rigid coil portion relative to the first rigid coil portion. Therefore, the first rigid coil portion and the second rigid coil portion move more slowly under an external force to improve the comfort of the user.

In yet another exemplary embodiment of the ankle coil, a second support face for supporting the sole of the foot is formed at the bottom of the second channel of the second rigid coil portion. Therefore, the second support face supports and is snugly fitted with the sole of the foot so as to improve the comfort of the user.

In yet another exemplary embodiment of the ankle coil, a first support face for supporting the shank is formed at the bottom of the first channel of the first rigid coil portion. Therefore, the first support face supports and is snugly fitted with the heel and the shank so as to improve the comfort of the user.

Preferred embodiments are described below with reference to the accompanying drawings in an explicit and comprehensible manner, and the above characteristics, technical features, advantages, and embodiments of the ankle coil are further described.

### BRIEF DESCRIPTION OF THE DRAWINGS/FIGURES

The following accompanying drawings merely illustratively, which describe and explain the present disclosure and are not intended to limit the scope of the present disclosure.

FIG. 1 is a schematic exploded view of the structure of an ankle coil, according to an exemplary embodiment.

FIG. 2 is a schematic assembled view of the structure of the ankle coil, according to an exemplary embodiment.

FIG. 3 is a further schematic exploded view of the structure of the ankle coil, according to an exemplary embodiment.

FIG. 4 is a schematic view of the ankle coil in a usage state, according to an exemplary embodiment.

FIG. 5 is a schematic diagram of the ankle coil in another usage state, according to an exemplary embodiment.

FIG. 6 is a schematic partial view of an ankle coil, according to another exemplary embodiment.

### LIST OF REFERENCE SIGNS

10 First coil portion
12 First channel
13 Notch
14 First rigid coil portion
15 First flexible coil portion
16 First support face
20 Second coil portion
22 Second channel
23 Notch
24 Second rigid coil portion
25 Second flexible coil portion
26 Second support face
27 Limiting portion
50 Hinge member
51 First connector
52 Hinge shaft
53 Second connector
60 Elastic member
70 Damping member
80 Positioning member
R Axis of rotation
a Adjustment angle.

### DETAILED DESCRIPTION

In order to more clearly understand the technical features, objectives, and effects of the present disclosure, the specific embodiments of the present disclosure are described with reference to the accompanying drawings, and in the drawings, the same number represents components of the same structure or of similar structures but the same function.

The word “exemplary” represents “serving as an instance, example or description” herein, and any illustration and embodiment described as “exemplary” herein should not be interpreted as a more preferred or more advantageous technical solution.

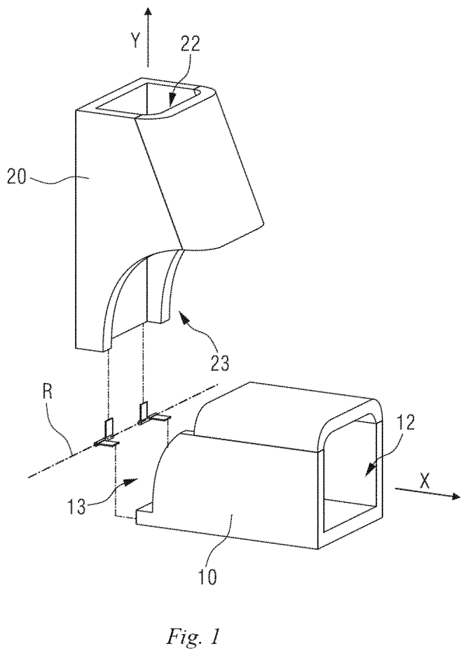

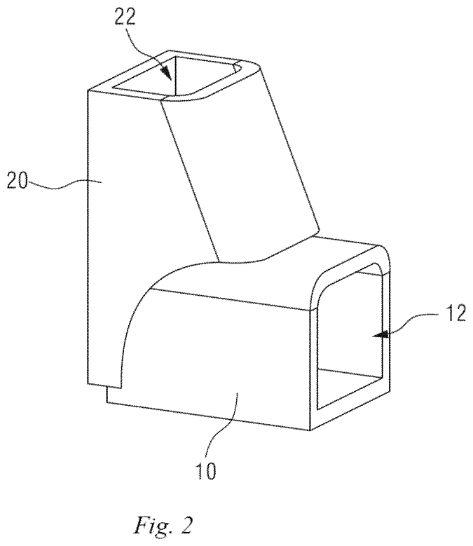

FIG. 1 is a schematic exploded view of the structure of an ankle coil, according to an exemplary embodiment. FIG. 2 is a schematic diagram of an assembled structure of the ankle coil. With reference to FIG. 1 and FIG. 2, the ankle coil comprises a first coil portion 10 and a second coil portion 20.

Specifically, the first coil portion 10 has a first channel 12 that is hollow and extends in a first extension direction X. The second coil portion 20 has a second channel 22 that is hollow and extends in a second extension direction Y intersecting with the first extension direction X. The second coil portion 20 is rotatably arranged on the first coil portion 10, with an axis of rotation R perpendicular to both the first extension direction X and the second extension direction Y, and a notch 13 and a notch 23 are respectively provided at a joint between the first coil portion 10 and the second coil portion 20. As shown in FIG. 1, the first coil portion 10 and the second coil portion 20 are hinged together by means of two hinges. Such a connection structure is simple, flexible, and easy to assemble. However, the present disclosure is not limited thereto; in other exemplary embodiments, only one hinge may be included, or the first coil portion 10 and the second coil portion 20 may be directly hinged together.

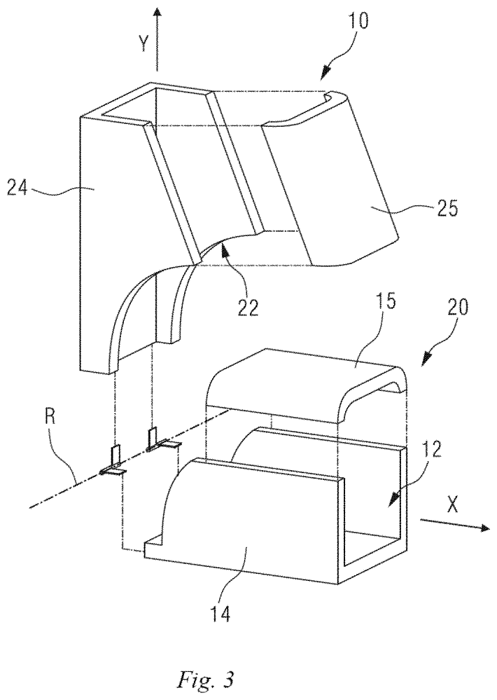

FIG. 3 is a schematic diagram of a further exploded structure of the ankle coil. Referring to FIG. 3, in an exemplary embodiment, the first coil portion 10 comprises a first rigid coil portion 14 and a first flexible coil portion 15. The first rigid coil portion 14 is formed with the first channel 12 having an opening on the side oriented in a direction of rotation thereof towards the second coil portion 20 for accommodating a heel and a shank, and a first support face 16 for supporting the heel and the shank is further formed at the bottom of the first channel 12 of the first rigid coil portion 14. The first flexible coil portion 15 is arranged on the first rigid coil portion 14 and is able to cover the side of the first channel 12 that is oriented in the direction of rotation.

The second coil portion 20 comprises a second rigid coil portion 24 and a second flexible coil portion 25. The second rigid coil portion 24 is rotatably arranged on the first rigid coil portion 14, and is formed with the second channel 22 having an opening opposite to the first channel 12 in the direction of rotation for accommodating the foot, and a second support face 26 for supporting the sole of the foot is formed at the bottom of the second channel 22 of the second rigid coil portion 24. The second flexible coil portion 25 is arranged on the second rigid coil portion 24 and is able to cover the side of the second channel 22 that is oriented in the direction of rotation. The first rigid coil portion 14, the first flexible coil portion 15, the second rigid coil portion 24, and the second flexible coil portion 25 are each provided with coil units for magnetic resonance imaging. The first flexible coil portion 15 and the second flexible coil portion 25 are separately formed by deformable flexible coils, and during use of the ankle coil, the first flexible coil portion 15 and the second flexible coil portion 25 can be deformed by pressing with an external force, to be snugly fitted with ankles of different sizes in the first channel 12 and the second channel 22, so that a better imaging effect can be obtained.

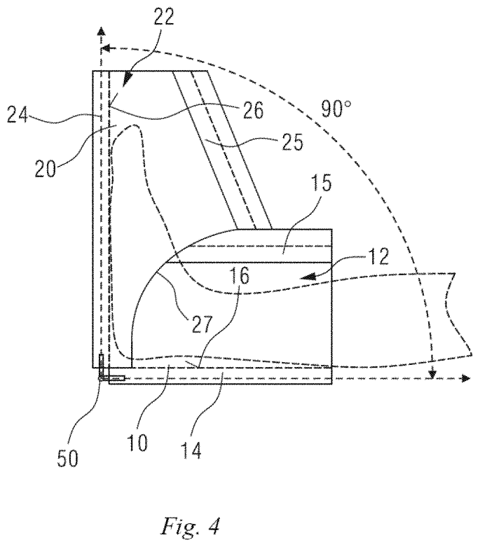

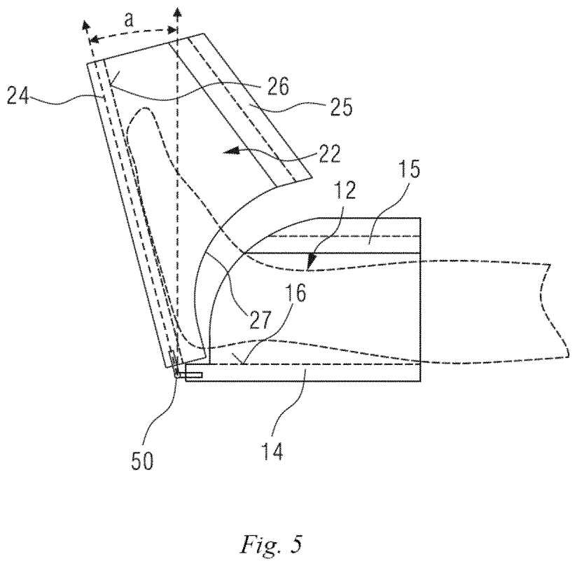

To enable those skilled in the art to better understand the present disclosure, a use process of the ankle coil in the present application is described in detail below. FIG. 4 and FIG. 5 are schematic diagrams of a use state of the ankle coil, wherein the first rigid coil portion 14 and the second rigid coil portion 24 cooperate with the first flexible coil portion 15 and the second flexible coil portion 25, respectively, to form accommodation spaces, one being used to accommodate the heel and the shank, and the other being used to accommodate the foot. In FIG. 4 and FIG. 5, dashed lines within the ankle coil represent inner surfaces of the ankle coil. After an ankle of a user is put into the ankle coil, generally, as shown in FIG. 4, the extension direction of the second channel 22 is perpendicular to the extension direction of the first channel 12, and in this case, the foot of the user is tightened. To enable the user to be relaxed, the second rigid coil portion 24 can rotate relative to the first rigid coil portion 14 by an adjustment angle a as shown in FIG. 5, wherein the adjustment angle ‘a’ is adjusted according to an actual situation of the user, so that the ankle coil can be snugly fitted with all parts of the ankle while making the ankle form a natural plantar flexion. In this case, the first flexible coil portion 15 and the second flexible coil portion 25 are further pressed so as to be snugly fitted with the instep and the shank bone, respectively. After the preparation, the ankle of the user can be scanned. The ankle coil can improve the comfort of the user while ensuring the imaging quality. In addition, the first flexible coil portion 15 and the second flexible coil portion 25 form integral structures with the first rigid coil portion 14 and the second rigid coil portion 24 respectively, which facilitates the connection arrangement of internal coils thereof.

In an exemplary embodiment, and referring to FIG. 4 and FIG. 5, the second rigid coil portion 24 has a limiting portion 27, and when the second rigid coil portion 24 rotates towards the first rigid coil portion 14 in the direction of rotation until the extension direction of the second channel 22 is perpendicular to the extension direction of the first channel 12, the limiting portion 27 bears against the first rigid coil portion 14 to prevent further rotation of the second rigid coil portion. Therefore, a case in which an angle between the first rigid coil portion 14 and the second rigid coil portion 24 is too small to facilitate the user to put the ankle into the ankle coil is avoided.

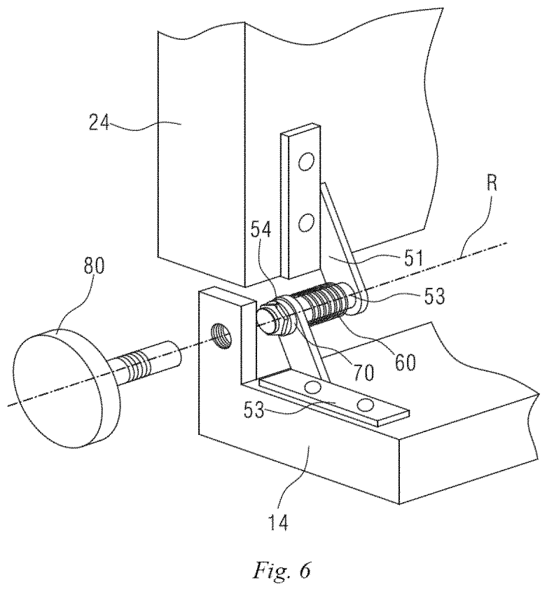

FIG. 6 is a partial schematic diagram of the ankle coil according to another exemplary embodiment. Referring to FIG. 6, the structure is the same as that in the exemplary embodiment shown in FIG. 1 and thus is not repeatedly described. The difference between the embodiments as shown in FIG. 1 and FIG. 6 lies in that the ankle coil as shown in FIG. 6 further comprises a hinge member 50 that replaces the hinge as shown in FIG. 1, and the hinge member as shown in FIG. 6 also comprises a first connector 51, a second connector 53, and a hinge shaft 52. The first connector 51 is fixed to the first rigid coil portion 14. The second connector 53 is fixed to the second rigid coil portion 24. The second connector 53 is fixed to the hinge shaft 52, the first connector 51 is rotatably sheathed on the hinge shaft 52, and an axial direction of the hinge shaft 52 coincides with the axis of rotation R.

As shown in FIG. 6, an end of the hinge shaft 52 is further provided with a nut 54 for limiting the first connector 51 on the hinge shaft 52, and therefore, a rotational connection between the first rigid coil portion 14 and the second rigid coil portion 24 is achieved by means of a simple structure. Those skilled in the art can understand that the hinge shaft 52 is not limited to being fixed to the second connector 53, and the hinge shaft can also be fixed to the first connector 51, and in order to achieve the rotational connection, the second connector 53 is rotatably sheathed on the hinge shaft 52.

In an exemplary embodiment, as shown in FIG. 6, the ankle coil further comprises a positioning member 80 which is arranged in the first rigid coil portion 14 so as to be movable along the axis of rotation R by means of a thread and can be fixed at any time. The positioning member bears against one end of the hinge shaft 52 along the direction of movement thereof, and fixes the hinge shaft 52 by means of a frictional force in order to further fix a position of the second rigid coil portion 24 relative to the first rigid coil portion 14. Therefore, movement artifacts caused by coil shaking due to involuntary movement of a patient are avoided. Those skilled in the art can understand that, the positioning member 80 is not limited to being arranged on the first rigid coil portion 14, it can also be arranged on the second rigid coil portion 24, and bear against and be fixed to the first connector 51 of the first rigid coil portion 14 or directly abut against the first rigid coil portion 14 along the direction of movement.

As shown in FIG. 6, the ankle coil further comprises an elastic member 60, and in an exemplary embodiment, the elastic member is a torsion spring, wherein one end of the torsion spring applies a force to the first rigid coil portion 14 by means of the first connector 51, and the other end applies a force to the second rigid coil portion 24 by means of the second connector 53 so as to continuously apply, to the second rigid coil portion 24, an elastic restoring force that rotates the second rigid coil portion towards the first rigid coil portion 14 in the direction of rotation. Therefore, the second rigid coil portion 24 can be automatically restored to a position at which the extension direction of the second channel 22 is perpendicular to the extension direction of the first channel 12 when no force is applied, so that the user can put the ankle into the ankle coil. Those skilled in the art can understand that, the elastic member 60 is not limited to the torsion spring, it can also be a tension spring with two ends that are respectively connected to the first rigid coil portion 14 and the second rigid coil portion 24, or other devices that can generate an elastic restoring force between the first rigid coil portion 14 and the second rigid coil portion 24.

Referring to FIG. 6, the ankle coil further comprises a damping member 70, and in an exemplary embodiment, the damping member is an elastic gasket which is sheathed on the hinge shaft 52 and located on one side of the second connector 53 for increasing the frictional force of rotation of the second connector 53 relative to the hinge shaft 52 to increase resistance against rotation of the second rigid coil portion 24 relative to the first rigid coil portion 14. Therefore, the simple structure enables the first rigid coil portion 14 and the second rigid coil portion 24 to move more slowly under an external force so as to improve the comfort of the user. However, the present disclosure is not limited thereto; in another exemplary embodiment, the damping member 70 can also be other devices that can generate resistance against movement between the first rigid coil portion 14 and the second rigid coil portion 24.

It should be understood that, although the specification is described according to various embodiments, each of the embodiments does not only contain one independent technical solution. This description of the specification is for the sake of clarity. Those skilled in the art should take the specification as a whole, and the technical solutions in the various embodiments may also be combined appropriately to form other embodiments that can be understood by those skilled in the art.

The series of detailed descriptions set forth above are merely specific illustrations of feasible embodiments of the present disclosure, and are not intended to limit the protection scope of the present disclosure. Equivalent embodiments or variations without departing from the technical spirit of the present disclosure, such as combinations, divisions, or repetitions of the features, should fall within the protection scope of the present disclosure.

What is claimed is:

1. An ankle coil configured to be worn by a patient during magnetic resonance imaging, the ankle coil comprising:
a first coil portion including a first channel extending in a first direction; and
a second coil portion including a second channel extending in a second direction that intersects with the first direction,
wherein a joint is formed between the first coil portion and the second coil portion via mating notches, and
wherein the second coil portion is rotatably arranged on the first coil portion with an axis of rotation perpendicular to both the first direction and the second direction.

2. The ankle coil of claim 1, wherein:
the first coil portion includes (i) a first rigid coil portion formed with the first channel having a first opening on a side of the first coil portion that is oriented towards the second coil portion in a direction of rotation, and (ii) a first flexible coil portion arranged on the first rigid coil portion, the first flexible coil portion covering the opening; and
the second coil portion includes (i) a second rigid coil portion rotatably arranged with respect to the first rigid coil portion, the second rigid coil portion being formed with the second channel having a second opening that is oriented opposite to the first channel in the direction of rotation, and (ii) a second flexible coil portion arranged on the second rigid coil portion, the second flexible coil portion covering the second opening.

3. The ankle coil of claim 2, wherein the first rigid coil portion, the first flexible coil portion, the second rigid coil portion, and the second flexible coil portion each include a respective coil unit configured for use with magnetic resonance imaging.

4. The ankle coil of claim 2, further comprising:
a hinge member including (i) a first connector that is fixed to the first rigid coil portion, (ii) a second connector that is fixed to the second rigid coil portion, and (iii) a hinge shaft,
wherein one of the first connector and the second connector is fixed to the hinge shaft, and
wherein the other one of the first connector and the second connector is rotatably sheathed on the hinge shaft, and
wherein an axial direction of the hinge shaft coincides with the axis of rotation.

5. The ankle coil of claim 2, further comprising:
a positioning member,
wherein the positioning member is arranged in one of the first rigid coil portion and the second rigid coil portion so as to be movable along a direction parallel to the axis of rotation, and
wherein the positioning member bears against the other of the first rigid coil portion and the second rigid coil portion along a direction of movement of the positioning member to captivate a position of the second rigid coil portion relative to the first rigid coil portion.

6. The ankle coil of claim 2, wherein the second rigid coil portion includes a limiting portion, and
wherein, when the second rigid coil portion is rotated towards the first rigid coil portion in the direction of rotation such that the direction of the second channel is perpendicular to the direction of the first channel, the limiting portion bears against the first rigid coil portion to prevent further rotation of the second rigid coil portion.

7. The ankle coil of claim 6, further comprising:
an elastic member,
wherein one end of the elastic member is arranged to apply a force to the first rigid coil portion, and
wherein another end of the elastic member is arranged to apply a force to the second rigid coil portion that rotates the second rigid coil portion towards the first rigid coil portion in the direction of rotation.

8. The ankle coil of claim 2, further comprising:
a damping member arranged between the first rigid coil portion and the second rigid coil portion, the damping member being configured to increase resistance to rotation of the second rigid coil portion relative to the first rigid coil portion.

9. The ankle coil of claim 2, further comprising:
a first support face arranged at the bottom of the first channel of the first rigid coil portion, the first support face being configured to support a shank.

10. The ankle coil of claim 9, further comprising:
a second support face arranged at the bottom of the second channel of the second rigid coil portion, the second support face being configured to support a sole of a foot.
